(19) United States
(12) Patent Application Publication
(10) Pub. No.: US 2014/0234220 A1
(43) Pub. Date: Aug. 21, 2014

(54) METHOD AND COMPOSITION FOR DISPERSIONS OF GOLD NANOPARTICLES

(71) Applicants: Dan V. Goia, Potsdam, NY (US); John I. Njagi, Potsdam, NY (US); Calin V. Pop, Brooksville, FL (US)

(72) Inventors: Dan V. Goia, Potsdam, NY (US); John I. Njagi, Potsdam, NY (US); Calin V. Pop, Brooksville, FL (US)

(21) Appl. No.: 14/183,229

(22) Filed: Feb. 18, 2014

Related U.S. Application Data
(60) Provisional application No. 61/765,506, filed on Feb. 15, 2013.

(51) Int. Cl. A61K 41/00; A61K 47/48

(52) U.S. Cl. CPC A61K 41/0042; A61K 47/48884
USPC 424/9.1; 514/769; 424/490

(57) ABSTRACT

The present disclosure relates to a method for making a gold nanoparticle dispersion. The method includes providing a first solution that has gold ions in a liquid medium, adding NTA molecules to the first solution to form a gold nanoparticle dispersion. When the NTA molecules are added to the first solution, the NTA molecules act as a reducing agent to reduce the gold ions to gold atoms, after which the gold atoms nucleate to form gold nanoparticles. The excess NTA molecules then attach to the surface of the gold nanoparticles to stabilize the dispersion. The present disclosure also relates to a dispersion composition that includes a liquid medium, gold nanoparticles dispersed throughout the liquid medium, and NTA molecules directly coating the gold nanoparticles.

### CROSS-REFERENCES TO RELATED APPLICATIONS

This application claims the benefit of U.S. Provisional Patent Application No. 61/765,506 entitled “METHOD FOR MAKING STABLE DISPERSIONS OF GOLD NANOPARTICLES” and filed on Feb. 15, 2013 for Dan V. Goia et al., which is incorporated herein by reference.

### FIELD

This invention relates to gold sols and more particularly to synthesizing gold dispersions.

### BACKGROUND

Gold nanoparticles have attracted substantial interest from scientists for over a century because of their unique physical, chemical, and surface properties. Dispersed gold nanoparticles can be implemented in a variety of applications, such as catalysis, non-linear optics, electronics, surface enhanced spectroscopy, pigments, biology, sensors, biosensors, drug delivery, dentistry, coatings, and DNA sequence detection, among others. Dispersed gold nanoparticles, often referred to as colloidal gold, can be manufactured in a variety of methods.

The majority of gold dispersions are prepared by using sodium citrate to reduce tetrachloroaurate (HAuCl₄). This method results in the synthesis of substantially spherical gold nanoparticles that are capped or covered with negatively charged citrate ions. Other wet chemical methods for formation of colloidal gold include the Brust method, the Perrault method, and the Martin method. The Brust method relies on reaction of chlorauric acid with tetraoctylammonium bromide in toluene and sodium borohydride. The Perrault method uses hydroquinone to reduce the HAuCl4 in a solution containing gold nanoparticle seeds. The Martin method uses reduction of HAuCl4 in water by NaBH4 with stabilizing agents (HCl and NaOH) present in a precise ratio. All of these conventional methods generally require the presence of dispersing agents to prevent the gold nanoparticles from aggregating and precipitating out of solution. Further, these methods generally are only capable of producing stable dispersions with a maximum gold concentration of about 0.3 mMols (milli-mols) per liter.

### SUMMARY

From the foregoing discussion, it should be apparent that a need exists for composition, system, and method that overcome the limitations of conventional colloidal gold manufacturing. Beneficially, such a composition, system, and method would provide a cost-effective method for producing stable and uniform gold nanoparticle dispersions that can be implemented and used in a variety of different applications.

The subject matter of the present application has been developed in response to the present state of the art, and in particular, in response to the problems and needs in the art that have not yet been fully solved by currently available colloidal gold manufacturing methods. Accordingly, the present disclosure has been developed to provide a gold nanoparticle dispersion composition and related manufacturing method that overcome many or all of the above-discussed shortcomings in the art.

The present disclosure relates to a method for making a gold nanoparticle dispersion. The method includes providing a first solution that has gold ions in a liquid medium. The method then includes adding NTA molecules to the first solution to form a gold nanoparticle dispersion. When the NTA molecules are added to the first solution, the NTA molecules act as a reducing agent to reduce the gold ions to gold atoms, after which the gold atoms nucleate to form gold nanoparticles. The excess NTA molecules then attach to the surface of the gold nanoparticles to stabilize the dispersion.

In one embodiment, the method for making a gold nanoparticle dispersion further includes functionalizing the attached NTA molecules. For example, functionalizing the attached NTA molecules may include attaching a functional constituent at the central nitrogen in the NTA molecules. In another embodiment, the gold nanoparticles may be at least partially directly functionalized by modifying or attaching constituents to the surface of the gold nanoparticles. In one embodiment, forming the gold nanoparticle dispersion occurs at a reaction temperature below about 100° C. The concentration of gold in the gold nanoparticle dispersion may be greater than about 0.3 mMols per liter. In another embodiment, the concentration of gold in the gold nanoparticle dispersion is greater than about 1.0 mMols per liter. In yet another embodiment, the concentration of gold in the gold nanoparticle dispersion is greater than about 1.5 mMols per liter.

The present disclosure also relates to a dispersion composition that includes a liquid medium, gold nanoparticles dispersed throughout the liquid medium, and NTA molecules directly coating the gold nanoparticles. As stated above, the concentration of gold in the gold nanoparticle dispersion may be greater than about 0.3 mMols per liter. In another embodiment, the concentration of gold in the gold nanoparticle dispersion is greater than about 1.0 mMols per liter. In yet another embodiment, the concentration of gold in the gold nanoparticle dispersion is greater than about 1.5 mMols per liter. The dispersion composition may further include functional constituents attached to the NTA molecules directly coating the gold nanoparticles.

According to one embodiment, the size of the individual gold nanoparticles is substantially uniform. The size of the gold nanoparticles may be between about 1 nm and 200 nm. In another embodiment, the shape of the individual gold nanoparticles may be spherical, lobular, elliptical, and platelet-like, among others.

In one embodiment, ratio of gold-to-NTA is between about 1:2 and 1:100. In another embodiment, the ratio of gold-to-NTA is between about 1:20 and 1:40.

The present disclosure further relates to a method for using a gold nanoparticle dispersion. The method includes providing a dispersion composition that has solid gold nanoparticles dispersed throughout a liquid medium. The solid gold nanoparticles have NTA molecules directly coating the solid gold nanoparticles. The method further includes functionalizing the NTA molecules to have a deliverable constituent and delivering the composition to a biological target. The method finally includes irradiating the composition to at least partially release the deliverable constituent. The method may further include functionalizing the attached NTA molecules.

The deliverable constituent may be a chemical agent that can be used in applications such as biological imaging, biological signaling, biological detection, immune and metabolic modification, microbiological applications, diagnostic and therapeutic organ targeting, gene-regulation, DNA regulation, RNA regulation, drug and nutraceutical delivery, drug modulation, therapy modulation (e.g., modification of effective dose and side effects), treatment of cancer, and other diagnostic and therapeutic applications.

Reference throughout this specification to features, advantages, or similar language does not imply that all of the features and advantages that may be realized with the present disclosure should be or are in any single embodiment of the invention. Rather, language referring to the features and advantages is understood to mean that a specific feature, advantage, or characteristic described in connection with an embodiment is included in at least one embodiment of the subject matter disclosed herein. Thus, discussion of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize that the subject matter of the present application may be practiced without one or more of the specific features or advantages of a particular embodiment. In other instances, additional features and advantages may be recognized in certain embodiments that may not be present in all embodiments of the disclosure. Further, in some instances, well-known structures, materials, or operations are not shown or described in detail to avoid obscuring aspects of the subject matter of the present disclosure. These features and advantages of the present disclosure will become more fully apparent from the following description and appended claims, or may be learned by the practice of the disclosure as set forth hereinafter.

### BRIEF DESCRIPTION OF THE DRAWINGS

In order that the advantages of the disclosure will be readily understood, a more particular description of the disclosure briefly described above will be rendered by reference to specific embodiments that are illustrated in the appended drawings. Understanding that these drawings depict only typical embodiments of the disclosure and are not therefore to be considered to be limiting of its scope, the subject matter of the present application will be described and explained with additional specificity and detail through the use of the accompanying drawings, in which:

FIG. 1 is a partial cross-sectional view of a schematic depiction of one embodiment of a dispersion composition that includes solid gold nanoparticles dispersed throughout a liquid medium with NTA molecules directly coating the solid gold nanoparticles;

FIG. 2 is a chart showing one embodiment of the reducing effect of the NTA in the dispersion composition;

FIG. 3A depicts TEM images showing various sizes of gold nanoparticles based on various gold-to-NTA ratios of the dispersion composition;

FIG. 3B is a chart showing various concentrations of gold in the dispersion composition, according to one embodiment;

FIG. 4 is a schematic flow chart diagram of one embodiment of a method for making stable dispersions of gold nanoparticles; and

FIG. 5 is a schematic flow chart diagram of one embodiment of a method for using stable dispersions of gold nanoparticles.

### DETAILED DESCRIPTION

Reference throughout this specification to “one embodiment,” “an embodiment,” or similar language means that a particular feature, structure, or characteristic described in connection with the embodiment is included in at least one embodiment of the present disclosure. Thus, appearances of the phrases “in one embodiment,” “in an embodiment,” and similar language throughout this specification may, but do not necessarily, all refer to the same embodiment. Similarly, the use of the term “implementation” means an implementation having a particular feature, structure, or characteristic described in connection with one or more embodiments of the present disclosure, however, absent an express correlation to indicate otherwise, an implementation may be associated with one or more embodiments.

Furthermore, the described features, structures, or characteristics of the disclosure may be combined in any suitable manner in one or more embodiments. In the following description, numerous specific details are provided. One skilled in the relevant art will recognize, however, that the subject matter of the present application may be practiced without one or more of the specific details, or with other methods, components, materials, and so forth. In other instances, well-known structures, materials, or operations are not shown or described in detail to avoid obscuring aspects of the disclosure.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

As described above, gold nanoparticle dispersions are useful because of their unique physical, chemical, and surface properties. A majority of the conventional methods for manufacturing gold dispersions generally require implementing a reducing agent and a separate dispersing agent, among other stabilizers and agents. Further, the majority of manufacturing methods produce gold nanoparticle dispersions that have a gold concentration of less than about 0.5 mMol per liter and/or that have gold particles with non-uniform sizes.

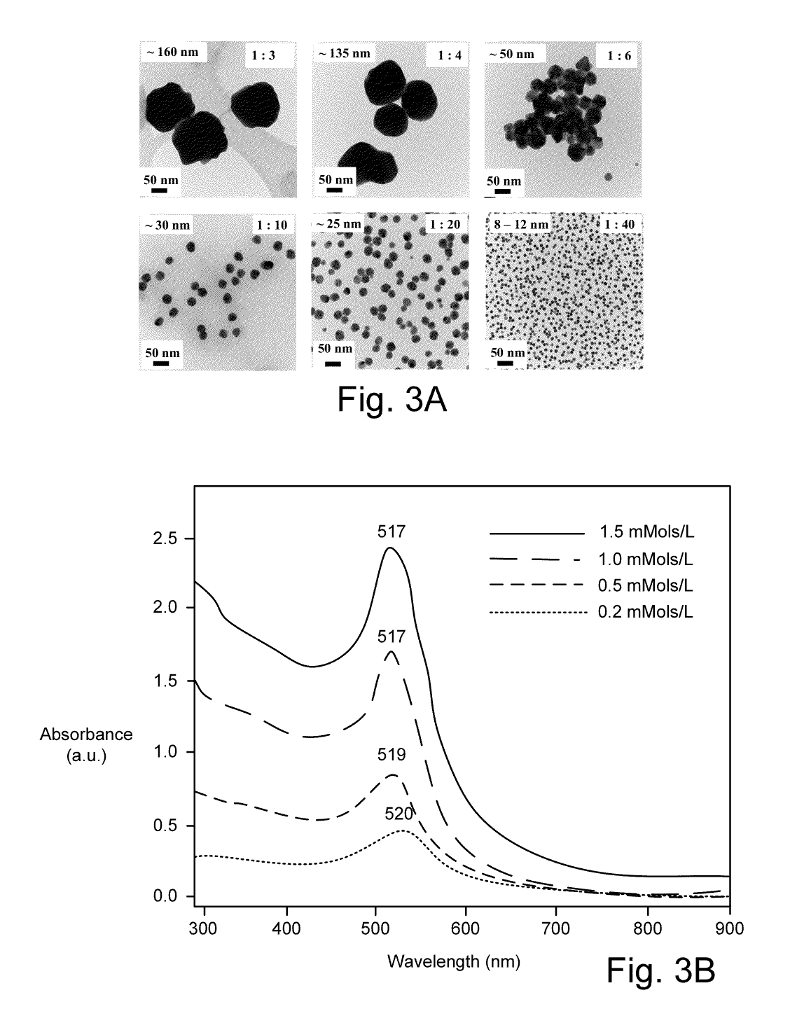

However, the subject matter of the present disclosure generally relates to a method for synthesizing a novel gold nanoparticle dispersion composition that is cost-effective and that has a comparatively higher stable maximum gold concentration. Additionally, the gold nanoparticle dispersion composition of the present disclosure can be functionalized according to a specific application (e.g., drug delivery). For example, using nitrotrilotriacetic acid (hereinafter “NTA”) as the reducing agent and the dispersing agent (described below with reference to FIGS. 3A-3B) offers more opportunities to functionalize the surface of the gold nanoparticles due to the presence of the nitrogen atom in the backbone of NTA. Also, the manufacturing method of the present disclosure can generate uniform dispersed gold nanoparticles in a comparatively broader size range (10-160 nm) and can yield dispersions with comparatively higher gold concentrations than conventional methods.

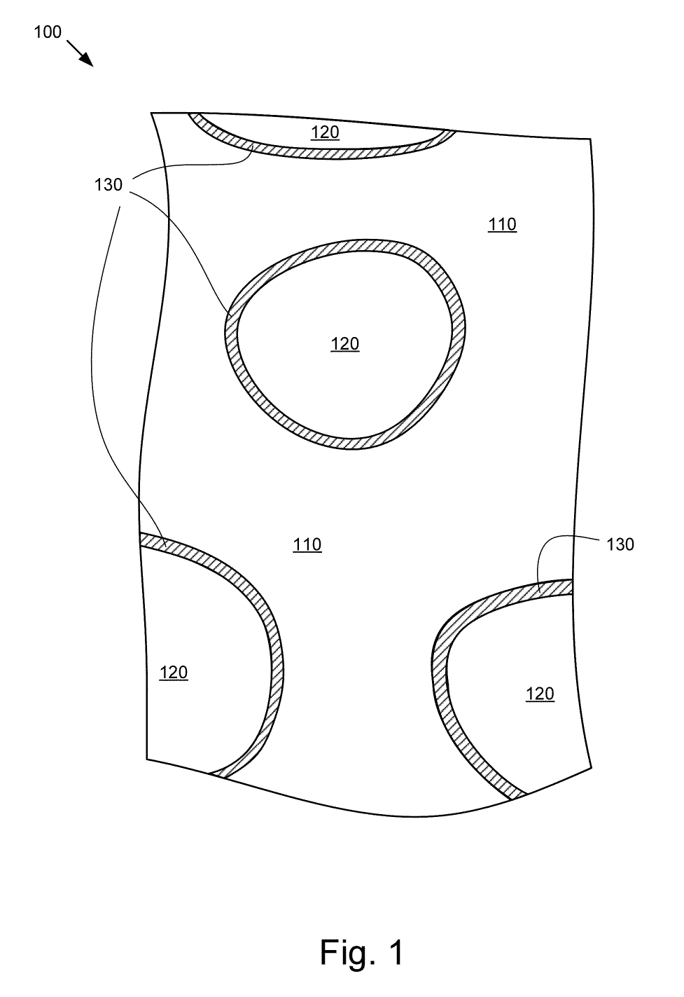

FIG. 1 is a partial cross-sectional view of a schematic depiction of one embodiment of a dispersion composition 100 that includes solid gold nanoparticles 120 dispersed throughout a liquid medium 110 with nitrilotriacetic acid molecules 130 directly coating the solid gold nanoparticles 120. Throughout the present disclosure, the term “dispersion composition” 100 refers to a colloidal composition, known as a sol, with solid gold nanoparticles 120 dispersed throughout the liquid medium 110.

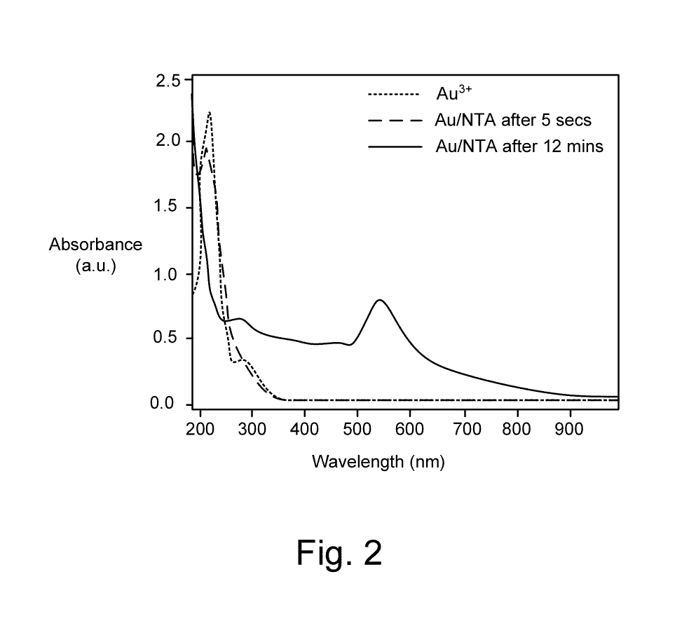

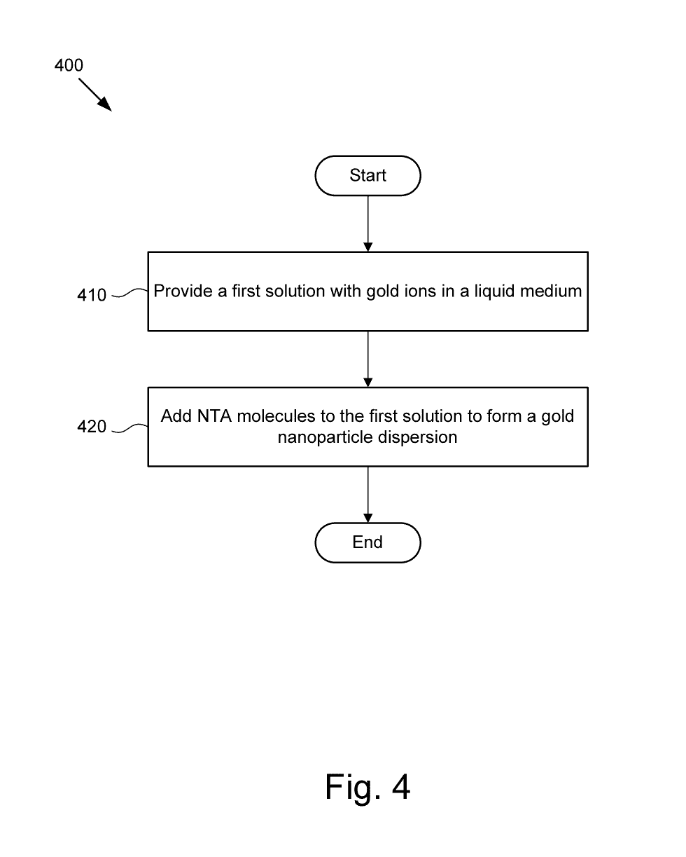

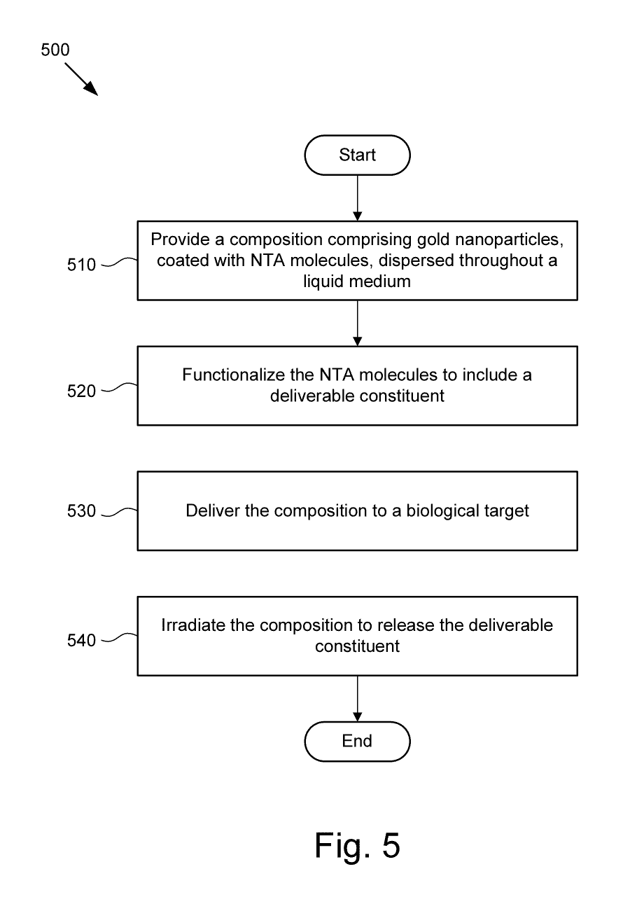

The various constituents 110, 120, 130 of the gold nanoparticle dispersion composition 100 are described below with reference to FIGS. 1-3B. Specifically, the liquid medium 110 is described immediately below, the gold nanoparticles are described below with reference to FIG. 2, and the NTA molecules 130 are described below with reference to FIGS. 2-3B. Additionally, details regarding a method for manufacturing the composition 100 and a method for using the composition 100 use are included below with reference to FIGS. 4-5, respectively. Generally, the gold nanoparticle dispersion composition 100 is stable at concentrations that are comparatively higher. For example, the concentration may be around an order of magnitude higher than conventional dispersions because of NTA's unique chemical properties as both a reducing agent and a dispersing agent.

In one embodiment, the liquid medium 110 is water. For example, the liquid medium 110 may be water that is purified via mechanical filters or chemical processes. To further purify the water used as the liquid medium 110, the water may be subjected to reverse osmosis, carbon filtration, ultraviolet oxidation, or electrodialysis, among other techniques. Further, the water may be distilled and/or de-ionized.

FIG. 2 is a chart showing one embodiment of the reducing effect that the NTA molecules have on the gold particles. As described in greater detail below, a precursor gold salt, such as tetrachloroauric acid (HAuCl₄) is combined with an NTA salt or an NTA hydrate. Ultimately the NTA molecules reduce the Au³⁺ to Au⁰. FIG. 2 shows an ultraviolet-visible spectroscopy absorbance rating for the constituents in the reduction process. Specifically, the dotted line represents the absorbance of the gold ions (Au³⁺), the dashed line represents the spectroscopy absorbance rating 5 seconds after the gold ions and the NTA have been combined together, and the solid line represents the spectroscopy absorbance rating 12 minutes after the gold ions and the NTA have been combined together. The NTA precursor is not shown in the chart of FIG. 2 to avoid obscuring the clarity of the disclosure because NTA alone does not register any noteworthy peaks in the depicted spectrum.

Electrons transfer from the NTA to the gold ion (Au³⁺) species, thereby causing the disappearance of the 218 and 289 nm absorption bands (dotted line, Au³⁺). The disappearance of the bands at 218 and 289 and the emergence of a new absorption band at 212 nm (dashed line) correspond to the pπ→5d×2−y2 and pσ→5d×2−y2 transitions in the square planar gold salt [AuCl₄⁻] complex ion. These changes suggest that the Au³⁺ ions are first reduced to Au¹⁺ to form a colorless Au(I)-NTA complex. The Au¹⁺ species are subsequently reduced by NTA to Au⁰ (gold atoms), as indicated by the disappearance of the Au(I)-NTA absorption band at 212 nm.

As the gold ions are reduced to gold atoms, gold nuclei begin to form as the concentration of gold atoms exceeds the critical supersaturation. As described below with reference to FIGS. 3A, and 3B, the gold nuclei grow by diffusional capture to form the final gold nanoparticles 120. The presence of the gold nanoparticles 120 is clearly indicated by the dispersion composition 100 turning a red color, as shown in FIG. 2 by the distinct absorption peak at 544 nm (solid line). The term “nanoparticle” as used in the context of the present disclosure refers to particles with a particle size in the range of less than about 200 nm as determined by conventional electron microscopy methods (SEM/TEM).

NTA is a biodegradable tetradentate trianionic ligand that has conventionally been used in water softening applications and as a replacement for sodium and potassium triphosphate in biologically friendly detergents. The NTA molecules that are used in the present disclosure may be derived from an NTA salt or an NTA hydrate. For example, nitrilotriacetic acid trisodium salt monohydrate may be used as the precursor to the NTA molecules. It is contemplated that the NTA molecules oxidize in two successive stages, with each stage resulting in the release of 2 electrons that are used to reduce the gold ions. The first oxidation stage converts NTA into imidodiacetic acid (IDA) and the second stage converts IDA into glycine (GLY). The glycine may further oxidize into ammonia, according to certain embodiments.

FIG. 3A depicts TEM images showing various sizes of gold nanoparticles 120 based on various gold-to-NTA ratios in the dispersion composition 100. As briefly described above, once the gold ions are reduced to gold atoms, the gold atoms begin to undergo nucleation. In non-colloidal environments, this nucleation would cause the solid gold to precipitate out of solution. However, as the gold atoms nucleate they form gold nanoparticles 120 and excess NTA molecules (i.e., the NTA molecules that are not oxidized during the reduction of the gold ions) are adsorbed onto the surface of the gold nanoparticles, thus functioning as a dispersing agent to stabilize the colloid.

A stable dispersion is one that will maintain the dispersed phase (e.g., gold nanoparticles) suspended in the liquid medium 110 substantially indefinitely. The stability of a dispersion is hindered by van der Waals attractive forces between the gold atoms that drive the gold atoms towards aggregation, precipitation, and sedimentation. Therefore, a stable dispersion will have repulsion forces that overcome the van der Waals attractive forces, thus preventing or limiting the continued aggregation of gold atoms and maintaining the nucleated gold nanoparticles in stable suspension. The two main mechanisms for preventing a dispersed phase from aggregating out of suspension are electrostatic stabilization and steric stabilization.

Electrostatic stabilization is the mutual repulsion of like charges and steric stabilization is the coating of the dispersed phase molecules to an extent such that the attractive forces between neighboring dispersed phase molecules is at least reduced. In the present disclosure, excess NTA molecules that were not consumed (oxidized) during the reduction of the gold ions attach to and coat the surface of the gold nanoparticles, thus likely providing steric stabilization to the dispersion composition 100. It is also contemplated that the attached NTA molecules also provide a degree of electrostatic stabilization because of the large negative zeta potential (ξ) of the gold nanoparticles coated with NTA (−56.2±1.4 mV).

Returning to FIG. 3A, various TEM images are depicted showing various sizes of gold nanoparticles 120 based on various gold-to-NTA ratios in the dispersion composition 100. The TEM images show the approximate and average size of the gold nanoparticles in the top left corner of each image, the ratio of gold-to-NTA molecules in the upper right corner of each image, and the scale in the bottom left corner of each image (all shown with a scale of 50 nm).

The trend shown in the TEM images suggests a gradual decrease in the size of the precipitated gold nanoparticles 120 as the concentration of NTA in the system increases. The TEM analysis shows that the comparatively lowest NTA concentration (1:3 ratio of gold to NTA) produced gold nanoparticles that were about 160 nm. On the other hand, the TEM analysis shows that the comparatively highest NTA concentration (1:40 ratio of gold to NTA) produced gold nanoparticles that were only about 10 nm in diameter. This trend is expected because an increasing reductant (NTA) concentration triggers a more rapid electron transfer, a faster nucleation, and thus the formation of smaller nanoparticles.

Also, the time required to attain stable optical properties decreases as the relative concentration of NTA increased (from 6 hours to 3 minutes for the gold-to-NTA ratios depicted in the TEM images). This is further indicative of the effect that the NTA molecules have on the reduction rate. Also noteworthy from the TEM images, the gold nanoparticles preserved their overall size uniformity for a very wide range of particle diameters. Therefore, regardless of the size of the gold nanoparticle required for a specific application, the dispersion composition 100 will have gold nanoparticles that are substantially stably dispersed throughout the liquid medium 110 and that have a substantially uniform size distribution.

FIG. 3B is a chart showing various concentrations of gold in the dispersion composition 100, according to one embodiment. All gold dispersions prepared at high excess of NTA (at least an NTA content of 1:5 ratio of gold to NTA) were stable substantially indefinitely. Once again, this stability is caused most likely by the steric and electrostatic stabilization imparted to the dispersion by the layer of NTA molecules coating the gold nanoparticles. The overall concentration of gold in the embodiments of the dispersion composition shown in FIGS. 2 and 3A was 0.2 mMols per liter. However, it is anticipated that the dispersion composition 100 will also be substantially stable at comparatively higher concentrations.

FIG. 3B specifically shows four different ultraviolet-visible spectra of dispersion compositions 100 with various overall concentrations of gold and with a gold-to-NTA ratio of 1:10. The bottom curve shows a gold concentration of 0.2 mMols per liter, the next curve up from the bottom shows a gold concentration of 0.5 mMols per liter, the next curve shows a gold concentration of 1.0 mMols per liter and the top curve shows a gold concentration of 1.5 mMols per liter. All of these dispersion compositions have a red-wine color (at least when the liquid medium 110 is water), as indicated by the absorption bands around 520 nm, and were stable for at least several months. The ultraviolet-visible spectra shown in FIG. 3B also shows narrow plasmon absorption bands of increasing intensity. The blue shift from 520 nm at the comparatively lowest gold concentration (0.2 mMols per liter) to 517 nm at the comparatively highest gold concentration (1.5 mMols per liter) indicates a likely decrease in particle size as the concentration of reactants increases.

In summary, the embodiments of the dispersion composition 100 described herein are stable at concentrations substantially higher than those produced via conventional preparation methods. Further, the manufacturing method (described below) of the present disclosure can generate substantially uniformly dispersed gold nanoparticles in a comparatively broader size range (1-200 nm). In another embodiment, the size range of the gold nanoparticles producable via the disclosed method may be 5-160 nm. Also, as described in greater detail below with reference to FIG. 4, the attached NTA molecules functioning as the dispersing agent offers comparatively more opportunities to functionalize the surface of the gold nanoparticles due to the presence of the nitrogen atom in the backbone of NTA.

FIG. 4 is a schematic flow chart diagram of one embodiment of a method 400 for making stable dispersions of gold nanoparticles. The method 400 includes providing 410 a first solution that has gold ions in a liquid medium. The gold ions may be the byproduct of a gold salt, such as tetrachloroaurate (HAuCl₄), disassociated in the liquid medium, such as de-ionized water. The method 400 further includes adding 420 NTA molecules (e.g., an NTA salt solution such as nitrilotriacetic acid trisodium salt monohydrate), to form a gold nanoparticle dipsersion. Forming the stable gold nanoparticle dispersion includes using the NTA molecules to reduce the gold ions to gold atoms, allowing the gold atoms to nucleate into gold nanoparticles, and using the excess NTA to attach to and coat the gold nanoparticles, thus forming a gold nanoparticle dispersion that is stabilized by the NTA. Depending on the relative gold-to-NTA ratio, the gold nanoparticles nucleate at a different rate and reach different size. The excess NTA molecules attach to and coat the gold nanoparticles, thereby stabilizing the dispersion and preventing the gold from precipitating or otherwise aggregating.

The temperature, mixing mechanism, mixing technique, and other such parameters are considered as secondary variables to the method and thus may be scaled/altered according to the specifics of a given application. The following example is one embodiment of a specific method for synthesizing a gold nanoparticle dispersion and is included in the present disclosure as an illustrative example and is not intended to limit the scope of the disclosure.

### Example

A concentrated solution of tetrachloroauric acid (HAuCl4, 23.11 wt. % Au) was selected as the gold salt. Nitrilotriacetic acid trisodium salt monohydrate (N(CH2CO2Na)3.H2O) [NTA] was selected as the NTA salt. A first solution containing 5.0 mMols per liter of gold and a second solution containing 20 mMols per liter of NTA were prepared by adding 2.13 grams of concentrated HAuCl₄ solution to 50 cm³ of de-ionized water and 2.75 grams of NTA to 50 cm³ of de-ionized (DI) water, respectively. A volume of 200 cm³ of de-ionized water was introduced into a 3-neck 500 cm³ spherical flask and heated to the reaction temperature. In one embodiment, the reaction temperature is between about 20° C. and 90° C. The first and second solutions were maintained at the same temperature and were added rapidly into the flask. The reaction mixture was stirred until the color and UV-Vis spectra of the dispersion stabilized.

The method 400 may further include functionalizing the NTA molecules that are attached to the gold nanoparticles. Once the coated gold nanoparticles are formed, additional stabilizing agents and/or biorecognization molecules may be attached to the NTA molecules. The functional constituents attached to the surface of the NTA molecules may include additional coating layers, functional ligands, and deliverable ligands, among others. In other words, the functional constituent may be configured to remain (potentially irreversibly) attached or partially attached to the NTA molecules or the functional constituent may be selected and configured to be at least partially removable under certain conditions (see the description below with reference to FIG. 5).

In one embodiment, gold nanoparticles with attached NTA that are surface-functionalized with ligands, such as biomolecules, may need to be dispersed into biological buffers to maintain the properties and functions of the biomolecules. However, biological buffers may weaken the electrostatic and steric stabilization forces of the attached NTA molecules. For example, electrolytes present in biological buffers may cause the negatively charged colloidal gold nanoparticles to draw together, aggregate, and to ultimately and irreversibly precipitate out of the suspension. Accordingly, the further surface modification may be required in order to maintain the stability of the gold dispersions for certain biological applications.

FIG. 5 is a schematic flow chart diagram of one embodiment of a method 500 for using stable dispersions of gold nanoparticles. The method 500 includes providing 510 a composition that has gold nanoparticles coated with NTA molecules that are dispersed throughout a liquid medium. The method 500 further includes functionalizing 520 the attached NTA molecules to include a functional constituent (e.g., a deliverable constituent). Still further, the method 500 includes delivering 530 the composition to a target area of a patient and irradiating 540 the composition to selectively release, or at least partially release, the deliverable constituent to the target area of the patient.

Depending on the specifics of a given application, the patient may be an animal, a human, biological tissue, or other living organism (“biological target”). The irradiation may be in the form of electromagnetic waves, heat, ultrasound, or other form of radiation. As described above, other methods of use may not involve removing the functional constituent but may involve irradiating to further modify, remove, and/or functionalize the attached constituent. Once again, the gold nanoparticles dispersion composition 100 of the present disclosure can be used in biomedical applications such as, but not limited to, biological imaging, biological signaling, biological detection, immune and metabolic modification, microbiological applications, diagnostic and therapeutic organ targeting, gene-regulation, DNA regulation, RNA regulation, drug and nutraceutical delivery, drug modulation, therapy modulation (e.g., modification of effective dose and side effects), treatment of cancer, and other diagnostic and therapeutic purposes, among others.

In the above description, certain terms may be used such as “up,” “down,” “upper,” “lower,” “horizontal,” “vertical,” “left,” “right,” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object. Further, the terms “including,” “comprising,” “having,” and variations thereof mean “including but not limited to” unless expressly specified otherwise.

An enumerated listing of items does not imply that any or all of the items are mutually exclusive and/or mutually inclusive, unless expressly specified otherwise. The terms “a,” “an,” and “the” also refer to “one or more” unless expressly specified otherwise. Further, the term “plurality” can be defined as “at least two.”

Additionally, instances in this specification where one element is “coupled” to another element can include direct and indirect coupling. Direct coupling can be defined as one element coupled to and in some contact with another element. Indirect coupling can be defined as coupling between two elements not in direct contact with each other, but having one or more additional elements between the coupled elements. Further, as used herein, securing one element to another element can include direct securing and indirect securing. Additionally, as used herein, “adjacent” does not necessarily denote contact. For example, one element can be adjacent another element without being in contact with that element.

As used herein, the phrase “at least one of”, when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example, without limitation, two of item A, one of item B, and ten of item C; four of item B and seven of item C; or some other suitable combination.

The subject matter of the present disclosure may be embodied in other specific forms without departing from its spirit or essential characteristics. The described embodiments are to be considered in all respects only as illustrative and not restrictive. The scope of the disclosure is, therefore, indicated by the appended claims rather than by the foregoing description. All changes which come within the meaning and range of equivalency of the claims are to be embraced within their scope.

What is claimed is:

1. A method for making a gold nanoparticle dispersion, the method comprising:
providing a first solution comprising gold ions in a liquid medium; and
adding NTA molecules to the first solution to form a gold nanoparticle dispersion.

2. The method of claim 1, wherein adding NTA molecules to the first solution to form a gold nanoparticle dispersion causes the NTA molecules to reduce the gold ions to gold atoms, the gold atoms to nucleate into gold nanoparticles, and the NTA molecules to coat the surface of the gold nanoparticles to stabilize the gold nanoparticle dispersion.

3. The method of claim 1, further comprising functionalizing the attached NTA molecules.

4. The method of claim 3, wherein functionalizing the attached NTA molecules comprises attaching a functional constituent at the nitrogen in the NTA molecules.

5. The method of claim 1, further comprising at least partially directly functionalizing the surface of the gold nanoparticles.

6. The method of claim 1, wherein the gold nanoparticle dispersion is stable substantially indefinitely.

7. The method of claim 1, wherein forming the gold nanoparticle dispersion occurs at a reaction temperature below about 100° C.

8. The method of claim 1, wherein the concentration of gold in the gold nanoparticle dispersion is greater than about 0.3 mMols per liter.

9. The method of claim 1, wherein the concentration of gold in the gold nanoparticle dispersion is greater than about 1.5 mMols per liter.

10. A dispersion composition comprising:
a liquid medium;
gold nanoparticles dispersed throughout the liquid medium; and
NTA molecules directly coating the gold nanoparticles.

11. The dispersion composition of claim 10, wherein the dispersion composition is stable substantially indefinitely.

12. The dispersion composition of claim 10, wherein the concentration of gold is greater than about 0.3 mMols per liter.

13. The dispersion composition of claim 10, wherein the concentration of gold is greater than about 1.5 mMols per liter.

14. The dispersion composition of claim 10, further comprising functional constituents attached to the NTA molecules directly coating the gold nanoparticles.

15. The dispersion composition of claim 10, wherein the size of the individual gold nanoparticles is substantially uniform.

16. The dispersion composition of claim 15, wherein the size of the gold nanoparticles is between about 1 nm and 200 nm.

17. The dispersion composition of claim 10, wherein the shape of the individual gold nanoparticles is selected from the group comprising spherical, lobular, elliptical, and platelet-like.

18. The dispersion composition of claim 10, wherein the ratio of gold-to-NTA is between about 1:20 and 1:40.

19. A method for using a gold nanoparticle dispersion, the method comprising:
providing a dispersion composition comprising solid gold nanoparticles dispersed throughout a liquid medium, wherein the solid gold nanoparticles have NTA molecules directly coating the solid gold nanoparticles;
functionalizing the NTA molecules to comprise a deliverable constituent;
delivering the composition to a biological target; and
irradiating the composition to at least partially release the deliverable constituent.

20. The method of claim 19, wherein the deliverable constituent is a chemical agent that can be used in applications such as biological imaging, biological signaling, biological detection, immune and metabolic modification, microbiological applications, diagnostic and therapeutic organ targeting, gene-regulation, DNA regulation, RNA regulation, drug and nutraceutical delivery, drug modulation, therapy modulation (e.g., modification of effective dose and side effects), treatment of cancer, and other diagnostic and therapeutic applications.
